(19) United States
(12) Patent Application Publication
(10) Pub. No.: US 2019/0275186 A1
(43) Pub. Date: Sep. 12, 2019

(54) SCENT DIFFUSER

(71) Applicant: DONGGUAN YIH TEH ELECTRIC PRODUCTS CO., LTD., Dongguan City (CN)

(72) Inventor: Ming Jen Hsiao, Miaoli County (TW)

(21) Appl. No.: 15/914,963

(22) Filed: Mar. 7, 2018

(51) Int. Cl. A61L 9/14

(52) U.S. Cl. CPC A61L 9/14; A61L 2209/134

(57) ABSTRACT

A scent diffuser contains: a body, a power connection unit, a pump, a spraying device, a gas tube, a sucking tube, and a connecting tube. The body includes an accommodation chamber and a first opening, the power connection unit is connected in the accommodation chamber, and the pump includes an air inflow segment and an air outflow segment. The spraying device is accommodated in the body and includes a bottle having a second opening, a bottom fence, and a lid. The gas tube is coupled in the bottle and has a shrinkage section, a channel, and a spout. The sucking tube is parallelly fixed in the bottle and has a vacuum suction mouth and a third opening, wherein a gap is defined between the third opening and the bottom fence. The connecting tube extends outward from the bottle and communicates with the gas tube and the connecting tube.

### FIELD OF THE INVENTION

The present invention relates to a scent diffuser which is simplified and has using safety.

### DESCRIPTION OF THE PRIOR ART

A conventional aroma diffuse is disclosed in U.S. Pat. No. 9,539,355, but its structure is complicated and has a large size. A conventional detachable aromatic nebulizing diffuser is disclosed in U.S. Pat. No. 7,963,460 and contains a fan configured to deliver scents toward an external space, but it consumes power of two electric devices. A lamp-based scent releasing system is disclosed in U.S. Pat. No. 8,201,957 and contains a heating element configured to heat aromatic substance, for example, an electric resistance heats the aromatic substance so as to diffuse scents, thus causing power consumption and heating danger.

The present invention has arisen to mitigate and/or obviate the afore-described disadvantages.

### SUMMARY OF THE INVENTION

The primary aspect of the present invention is to provide a scent diffuser which is simplified and has using safety.

To obtain the above aspect, a scent diffuser provided by the present invention contains: a body, a power connection unit, a pump, a spraying device, a gas tube, a sucking tube, and a connecting tube.

The body includes an accommodation chamber defined in the body and a first opening formed on a top of the accommodation chamber.

The power connection unit is connected in the accommodation chamber of the body.

The pump includes an air inflow segment and an air outflow segment, and the pump is housed in the accommodation chamber and is electrically connected with the power connection unit so as to pump gas.

The spraying device is accommodated in an upper end of the body, and the spraying device includes a bottle which has a second opening, a bottom fence, and a lid, wherein the lid has a first orifice and is configured to cover the second opening, the first orifice is in communication with the bottle and an exterior environment, and the bottle is configured to accommodate aromatic liquid.

The gas tube is coupled in the bottle and has a shrinkage section, a channel, and a spout, wherein the channel extends outward to the spout from a top of the shrinkage section.

The sucking tube is parallel to the gas tube and is fixed in the bottle, wherein the sucking tube has a vacuum suction mouth defined on a first free end thereof and located adjacent to the spout, a third opening formed on a second free end of the sucking tube, wherein a gap is defined between the third opening and the bottom fence of the bottle.

The connecting tube extends outward from the bottle, wherein a first end of the connecting tube communicates with the gas tube and a second end of the connecting tube communicates with the air outflow segment of the pump.

In operation, the aromatic liquid (or water) is fed into the second opening of the bottle, and the gas is successively delivered through the shrinkage section and the channel of the gas tube and is sprayed out of the spout by way of venturi effect which means successively delivering the gas through a narrowest portion of a tube at a highest flow speed so that a flow speed of the gas increases because of a change of a cross-sectional area of the tube (such as the shrinkage section) and a pressure decreases. Thereby, when the gas flows into the spout via the channel and sprays out of the spout via the vacuum suction mouth of the sucking tube, a pressure difference of the gas produces around the vacuum suction mouth so that the aromatic liquid is sucked to the vacuum suction mouth from the third opening of the gas tube and is sprayed to using space via the first orifice of the lid by the gas pumped by the spout, thus producing scents.

Accordingly, the scent diffuser sprays the scents by using the spraying device so as to produce the scents easily and safely. Preferably, the lid is removable so as to supplement the aromatic liquid or the water from the second opening, and the lid covers the second opening quickly.

### BRIEF DESCRIPTION OF THE DRAWINGS

FIG. 1 is a perspective view showing the assembly of a scent diffuser according to a preferred embodiment of the present invention.

FIG. 2 is a perspective view showing the exploded components of the scent diffuser according to the preferred embodiment of the present invention.

FIG. 3 is a top plan view showing the assembly of the scent diffuser according to the preferred embodiment of the present invention.

FIG. 4 is a cross sectional view taken along the line A-A of FIG. 3.

FIG. 5 is a cross sectional view taken along the line B-B of FIG. 3.

### DETAILED DESCRIPTION OF THE PREFERRED EMBODIMENTS

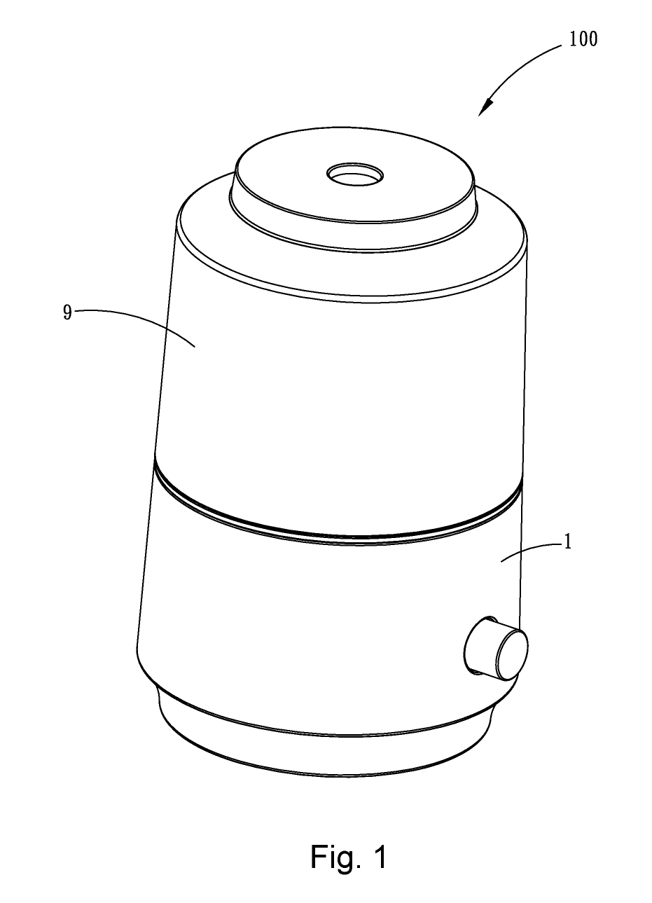

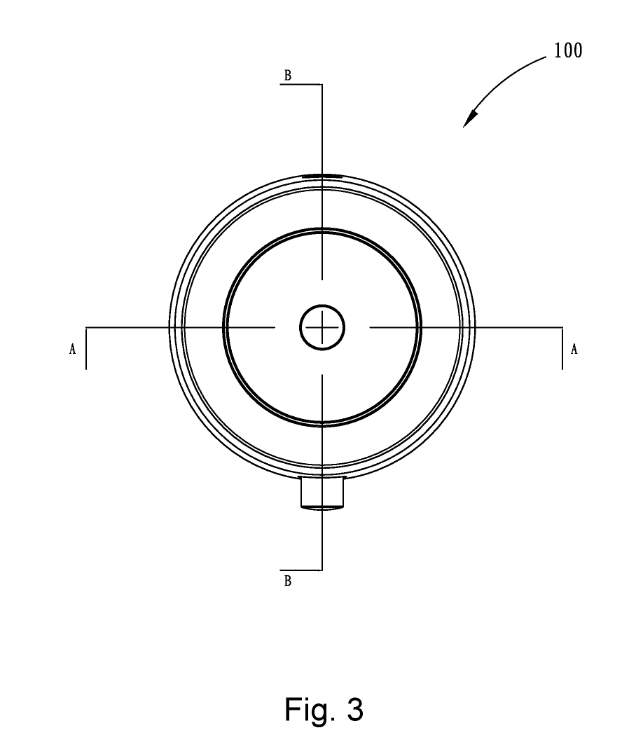

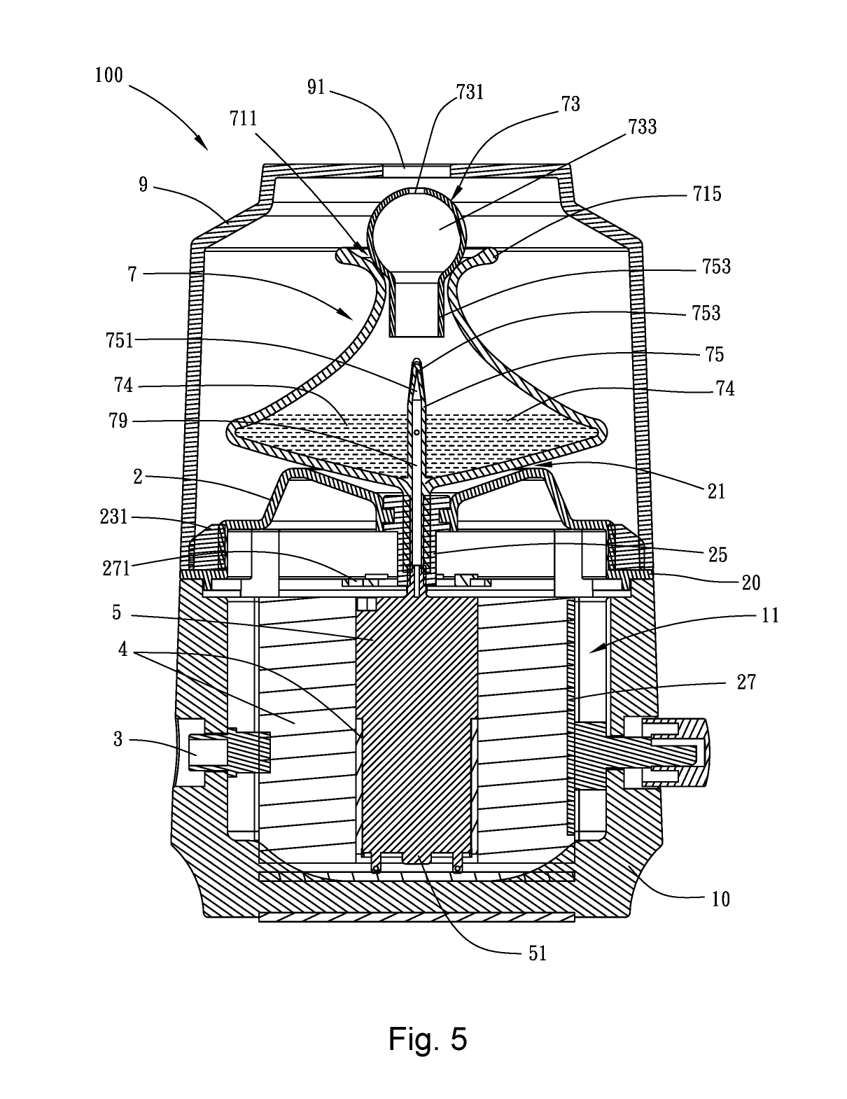

With reference to FIGS. 1-5, a scent diffuser 100 according to a preferred embodiment of the present invention comprises: a body 1 including an accommodation chamber 11 defined in the body 1 and a first opening 13 formed on a top of the accommodation chamber 11; a power connection unit 3 connected in the accommodation chamber 11 of the body 1, a pump 5 including an air inflow segment 51 and an air outflow segment 53, the pump 5 being housed in the accommodation chamber 11 and electrically connected with the power connection unit 3 so as to pump gas successively;

a spraying device 7 accommodated in an upper end of the body 1, and the spraying device 7 including a bottle 71 which has a second opening 711, a bottom fence 712, and a lid 73, wherein the lid 73 has a first orifice 731 and is configured to cover the second opening 711, the first orifice 731 is in communication with the bottle 71 and exterior environment, and the bottle 71 is configured to accommodate aromatic liquid 74; a gas tube 75 coupled in the bottle 71 and having a shrinkage section 751, a channel 753, and a spout 755, wherein the channel 753 extends outward to the spout 755 from a top of the shrinkage section 751; a sucking tube 77 being parallel to the gas tube 75 and fixed in the bottle 71, wherein the sucking tube 77 has a vacuum suction mouth 771 defined on a first free end thereof and located adjacent to the spout 755, a third opening 773 formed on a second free end of the sucking tube 77, wherein a gap is defined between the third opening 773 and the bottom fence 712 of the bottle 71; a connecting tube 79 extending outward from the bottle 71, wherein a first end of the connecting tube 79 communicates with the gas tube 75 and a second end of the connecting tube 79 communicates with the air outflow segment 53 of the pump 5 so as to receive the gas from the pump 5.

In operation, the aromatic liquid 74 (or water) is fed into the second opening 711 of the bottle 71, and the gas is successively delivered through the shrinkage section 751 and the channel 753 of the gas tube 75 and is sprayed out of the spout 755 by way of venturi effect which means successively delivering the gas through a narrowest portion of a tube at a highest flow speed so that a flow speed of the gas increases because of a change of a cross-sectional area of the tube (such as the shrinkage section 751) and a pressure decreases. Thereby, when the gas flows into the spout 755 via the channel 753 and sprays out of the spout 755 via the vacuum suction mouth 771 of the sucking tube 77, a pressure difference of the gas produces around the vacuum suction mouth 771 so that the aromatic liquid 74 is sucked to the vacuum suction mouth 771 from the third opening 773 of the gas tube 75 and is sprayed to using space via the first orifice 731 of the lid 73 by the gas pumped by the spout 755, thus producing scents.

Accordingly, the scent diffuser 100 sprays the scents by using the spraying device 7 so as to produce the scents easily and safely. Preferably, the lid 73 is removable so as to supplement the aromatic liquid 74 or the water from the second opening 711, and the lid 73 covers the second opening 711 quickly.

The aromatic liquid 74 is any one of essential oil, fragrance oil, essence, flower essence, perfume, and fragrance agent and is mixed with the water so as to produce the scents quickly after being atomized.

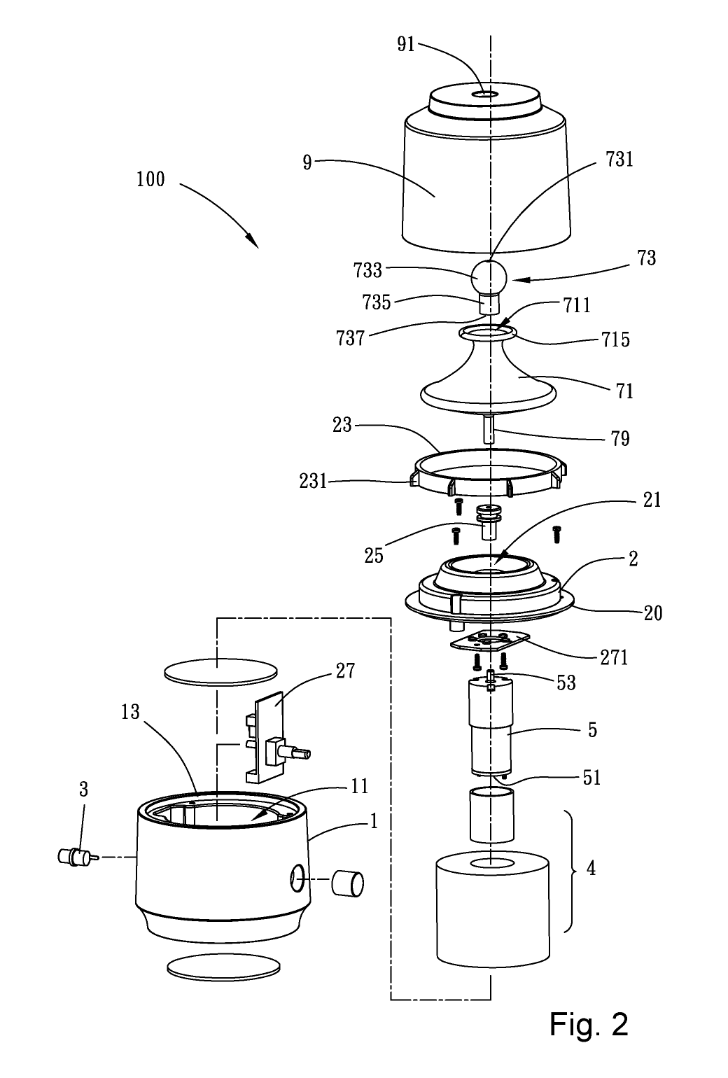

Referring to FIGS. 2, 4 and 5, a shrinkage tube structure 775 is arranged on an upper end of the sucking tube 77 and has the vacuum suction mouth 771 formed on a narrowest portion of the shrinkage tube structure 775, such that the shrinkage tube structure 775 urges the aromatic liquid 74 to spray out of the vacuum suction mouth 771 quickly so as to facilitate atomization of the scents by mating with the gas of the spout 755.

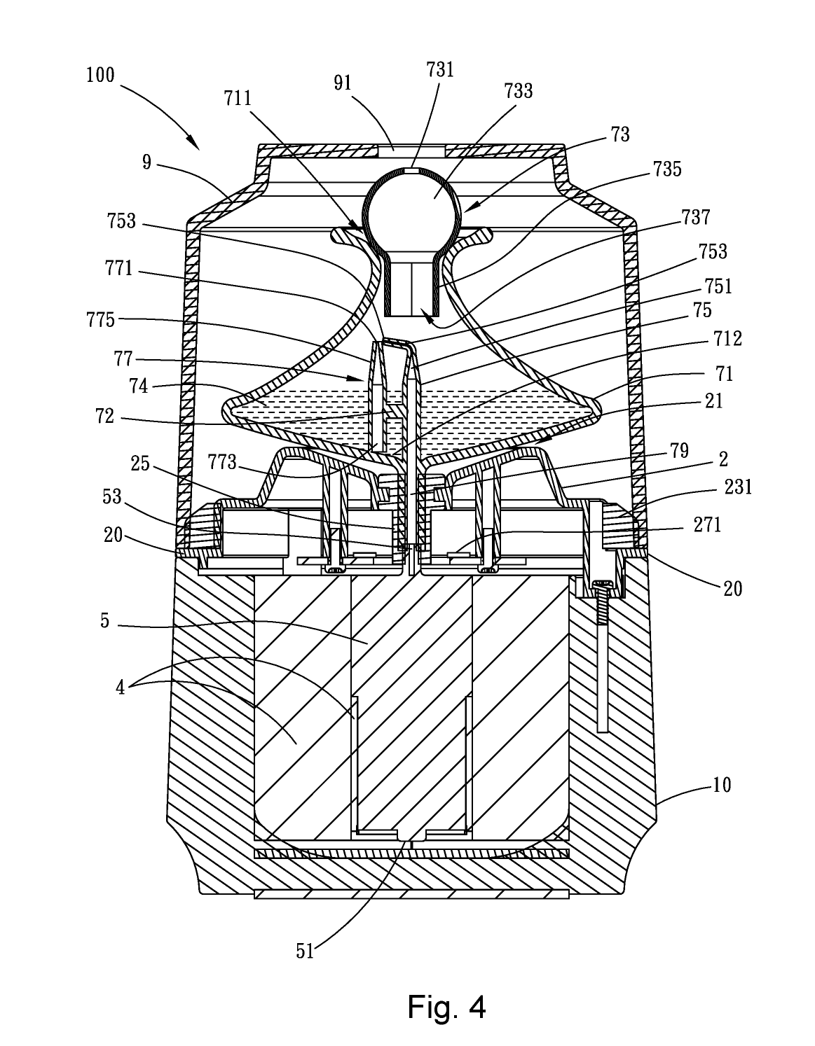

As shown in FIGS. 2 and 4, a coupling section 72 is defined between the sucking tube 77 and the gas tube 75, and the sucking tube 77 is parallelly connected with the gas tube 75 in the bottle 71 by way of the coupling section 72.

As illustrated in FIGS. 2, 4, and 5, the spout 755 of the gas tube 75 is proximate to and is higher than the vacuum suction mouth 771 of the sucking tube 77 so that the gas blows the aromatic liquid 74 above the vacuum suction mouth 771, thus atomizing the aromatic liquid 74.

With reference to FIGS. 2 and 4, the second opening 711 has a surround loop 715 formed around a peripheral side thereof, and the lid 73 has a spherical member 733 and a pipe 735, a first end of which communicates with the spherical member 733, and the pipe 735 has a second orifice 737 defined on a second end thereof, such that the lid 73 covers the second opening 711, the spherical member 733 is movably accommodated inside the surround loop 715, and the pipe 735 is housed into the bottle 71 and pulls the spherical member 733 downward so that the lid 73 is fixed inside the surround loop 715 securely so as to cover the second opening 711 and is removable from the second opening 711 easily.

With reference to FIGS. 2, 4, and 5, the scent diffuser 100 further comprises: a holder 2 including a through hole 21 defined on a center of the holder 2, and the holder 2 being connected on the first opening 13 of the body 1, wherein the air outflow segment 53 of the pump 5 inserts into the through hole 21, the second end of the connecting tube 79 inserts through the through hole 21 and communicates with the air outflow segment 53 of the pump 5 so that the pump 5 is in communication with the connecting tube 79 via the holder 2.

Referring to FIGS. 2, 4, and 5, the scent diffuser 100 further comprises a case 9 and a support ring 23, wherein the case 9 is hollow and includes a nozzle 91 formed on a top thereof, and the support ring 23 has multiple abutting wings 231 arranged around an outer wall thereof, wherein the support ring 23 is fitted on an outer wall of the holder 2. When the case 9 covers on the outer wall of the holder 2, an inner wall of the case 9 is removably retained on the multiple abutting wings 231 of the support ring 23, hence the support ring 23 is configured to secure the case 9, and the case 9 is configured to open or cover the holder 2. Preferably, the support ring 23 is made of flexible material, such as silicone, rubber or plastic.

Referring to FIGS. 2-4, the holder 2 further includes a hollow fixing tube 25 housed in the through hole 21 of the holder 2 and made of flexible material, wherein the flexible material is made of any one of silicone, rubber, and elastic cotton, such that the air outflow segment 53 of the pump 5 inserts into a lower end of the hollow fixing tube 25, the second end of the connecting tube 79 inserts through the hollow fixing tube 25 so as to communicate with the air outflow segment 53 of the pump 5, and the hollow fixing tube 25 secures and protects the connecting tube 79 and the air outflow segment 53 of the pump 5 in the through hole 21.

As shown in FIGS. 2, 4 and 5, the scent diffuser 100 further comprises a circuit board 27 and a light emitting element 271. The light emitting element 271 is electrically connected with the circuit board 27 which is electrically connected with the power connection unit 3, wherein the circuit board 27 and the light emitting element 271 are connected on a bottom of the holder 2, and the holder 2 is made of transparent material or translucent material. The holder 2 includes a light guide loop 20 formed on the outer wall thereof, such that when the case 9 covers the outer wall of the holder 2, a bottom of the case 9 retains on the light guide loop 20, and the upper end of the body 1 is located on a bottom of the light guide loop 20, such that the power connection unit 3 is coupled with a power supply so as to supply power toward the circuit board 27, by which the light emitting element 271 illuminates lights, and the holder 2 receives the lights and the light guide loop 20 displays the lights outside the scent diffuser 100, thus obtaining decoration effect.

Referring to FIGS. 2, 4, and 5, the scent diffuser 100 further comprises a sound absorbing cotton 4 accommodated between the pump 5 and the accommodation chamber 11 of the body 1 so as to absorb noises.

Accordingly, the scent diffuser is simplified and atomizes the aromatic liquid immediately so as to spray the scents quickly and safely.

While the preferred embodiments of the invention have been set forth for the purpose of disclosure, modifications of the disclosed embodiments of the invention as well as other embodiments thereof may occur to those skilled in the art. The scope of the claims should not be limited by the preferred embodiments set forth in the examples, but should be given the broadest interpretation consistent with the description as a whole.

What is claimed is:

1. A scent diffuser comprising:
a body including an accommodation chamber defined in the body and a first opening formed on a top of the accommodation chamber;
a power connection unit connected in the accommodation chamber of the body;
a pump including an air inflow segment and an air outflow segment, the pump being housed in the accommodation chamber and electrically connected with the power connection unit so as to pump gas;
a spraying device accommodated in an upper end of the body, and the spraying device including a bottle which has a second opening, a bottom fence, and a lid, wherein the lid has a first orifice and is configured to cover the second opening, the first orifice is in communication with the bottle and an exterior environment, and the bottle is configured to accommodate aromatic liquid;
a gas tube coupled in the bottle and having a shrinkage section, a channel, and a spout, wherein the channel extends outward to the spout from a top of the shrinkage section;
a sucking tube being parallel to the gas tube and fixed in the bottle, wherein the sucking tube has a vacuum suction mouth defined on a first free end thereof and located adjacent to the spout, a third opening formed on a second free end of the sucking tube, wherein a gap is defined between the third opening and the bottom fence of the bottle; and
a connecting tube extending outward from the bottle, wherein a first end of the connecting tube communicates with the gas tube and a second end of the connecting tube communicates with the air outflow segment of the pump.

2. The scent diffuser as claimed in claim 1, wherein a shrinkage tube structure is arranged on an upper end of the sucking tube and has the vacuum suction mouth formed on a narrowest portion of the shrinkage tube structure.

3. The scent diffuser as claimed in claim 2, wherein a coupling section is defined between the sucking tube and the gas tube, and the sucking tube is parallelly connected with the gas tube in the bottle by way of the coupling section.

4. The scent diffuser as claimed in claim 1, wherein the second opening has a surround loop formed around a peripheral side thereof, and the lid has a spherical member and a pipe, a first end of which communicates with the spherical member, and the pipe has a second orifice defined on a second end thereof, such that the lid covers the second opening, the spherical member is movably accommodated inside the surround loop, and the pipe is housed into the bottle.

5. The scent diffuser as claimed in claim 1 further comprises a holder including a through hole defined on a center of the holder, and the holder being connected on the first opening of the body, wherein the air outflow segment of the pump inserts into the through hole, the second end of the connecting tube inserts through the through hole and communicates with the air outflow segment of the pump.

6. The scent diffuser as claimed in claim 5 further comprising a case and a support ring, wherein the case is hollow and includes a nozzle formed on a top thereof, and the support ring has multiple abutting wings arranged around an outer wall thereof, wherein the support ring is fitted on an outer wall of the holder, when the case covers on the outer wall of the holder, an inner wall of the case is removably retained on the multiple abutting wings of the support ring.

7. The scent diffuser as claimed in claim 5, wherein the holder further includes a hollow fixing tube housed in the through hole of the holder, the air outflow segment of the pump inserts into a lower end of the hollow fixing tube, and the second end of the connecting tube inserts through the hollow fixing tube so as to communicate with the air outflow segment of the pump.

8. The scent diffuser as claimed in claim 6 further comprising a circuit board and a light emitting element; wherein the light emitting element is electrically connected with the circuit board which is electrically connected with the power connection unit, wherein the circuit board and the light emitting element are connected on a bottom of the holder, and the holder is made of transparent material or translucent material, and the holder includes a light guide loop formed on the outer wall thereof, such that when the case covers the outer wall of the holder, a bottom of the case retains on the light guide loop, and the upper end of the body is located on a bottom of the light guide loop.

9. The scent diffuser as claimed in claim 1 further comprising a sound absorbing cotton accommodated between the pump and the accommodation chamber of the body.

10. The scent diffuser as claimed in claim 1, wherein the spout of the gas tube is higher than the vacuum suction mouth of the sucking tube.
